(19) United States
(12) Patent Application Publication
(10) Pub. No.: US 2024/0315833 A1
(43) Pub. Date: Sep. 26, 2024

(54) Shape-Shifting Intraocular Display System

(71) Applicant: Ceyeber Corp., Newport Beach, CA (US)

(72) Inventor: Robert Edward Grant, Laguna Beach, CA (US)

(21) Appl. No.: 18/125,648

(22) Filed: Mar. 23, 2023

(51) Int. Cl. A61F 2/16; G06T 5/00; G09G 3/00

(52) U.S. Cl. CPC A61F 2/1648; A61F 2/1635; G06T 5/80; G09G 3/035; G09G 2320/02; G09G 2340/0407; G09G 2340/14

(57) ABSTRACT

A system for displaying intraocular images that includes a shape-adjustable lens with a transparent display disposed on the lens. When the shape of the lens changes, the transparent display's shape also changes, causing an image to be distorted. The system detects the change in the shape of the lens or the display and corrects the image accordingly so that it is once again properly displayed and viewed by the wearer of the system.

### FIELD OF THE INVENTION

The field of the invention is intraocular display systems.

### BACKGROUND

The background description includes information that may be useful in understanding the present invention. It is not an admission that any of the information provided herein is prior art or relevant to the presently claimed invention, or that any publication specifically or implicitly referenced is prior art.

As technology becomes smaller and smaller, medical procedures that were only recently purely science-fiction are becoming possible every passing day.

One field that has experienced rapid growth and development is intraocular implants. Deterioration of the eye due to age, disease or physical trauma used to mean partial or full blindness. Now, thanks to advances in this technology, intraocular implants have enabled patients to maintain or even regain eye function that was previously lost.

For example, U.S. Pat. No. 9,454,021 to Guillon discloses an intraocular lens that can alter its shape to allow the user to focus at different distances, mirroring the function of a fully-enabled eye.

Another field that has seen exciting developments is intraocular display systems. These miniature display devices allow for the user to view information intra-ocularly, allowing for great immersion in augmented reality environments and to access information in an inconspicuous and private manner.

One example of such a system is Applicant's own application Ser. No. 17/103,309 filed May 26, 2022 and titled “Augmented Reality Using Intra-Ocular Devices”.

Unfortunately, current systems do not allow for the accommodation of both intraocular aids and display devices in a single solution. There is limited space inside the eye for separate systems and the nature of a shape-changing lens introduces problems for a display system. For example, changes in the shape of the lens can cause distortion in a displayed image.

Thus, there is still a need an intraocular implant that enables the benefits of an intraocular display to intraocular aids as shape-adjusting intraocular lenses.

### SUMMARY OF THE INVENTION

The inventive subject matter provides apparatus, systems and methods in which a system for displaying intraocular images can adjust the display of an image based on a change in the shape of a shape-adjustable lens.

A system includes a shape-adjustable lens having a transparent display that is configured to display imagery. The display can be disposed on a rear side of the shape-adjustable lens or on the front side of the shape-adjustable lens. In embodiments, the display is internal to the shape-adjustable lens.

A processor of the system is programmed to cause the transparent display to display an image. When the shape of the shape-adjustable lens changes, the processor detects the change in the shape of the shape-adjustable lens (and consequently, a distortion of the displayed image), it adjusts the display of the image based on the detected change to continue to provide a proper image via the transparent display.

The adjustment can be based on a distortion to the proportions of the image, a distortion to the shape of the image, among others. Thus, in embodiments, the adjustment itself can be a change of the shape and/or proportions of the displayed image such that the image appears normally once again. In embodiments, the adjustment can include an adjustment to the resolution of the image being displayed.

Various objects, features, aspects and advantages of the inventive subject matter will become more apparent from the following detailed description of preferred embodiments, along with the accompanying drawing figures in which like numerals represent like components.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a.” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### BRIEF DESCRIPTION OF THE DRAWING

FIG. 1 shows a diagrammatic overview of a system deployed within a user's eye, according to embodiments of the inventive subject matter.

FIG. 2A shows a perspective from the view of the user of the shape-adjustable lens at its default shape with the transparent display showing an image.

FIG. 2B shows a side view of the lens of FIG. 2A.

FIG. 2C shows that the change in the shape of the lens and the corresponding change in the shape of the surface of the display has caused the image to become distorted.

FIG. 2D shows the side view of the thickened lens with the distorted display.

FIG. 3 is a flowchart that illustrates the processes to correct a distorted or otherwise changed image, executed according to embodiments of the inventive subject matter.

FIG. 4 illustrates a correction of a distorted image, according to embodiments of the inventive subject matter.

### DETAILED DESCRIPTION

Throughout the following discussion, numerous references will be made regarding servers, services, interfaces, engines, modules, clients, peers, portals, platforms, or other systems formed from computing devices. It should be appreciated that the use of such terms, is deemed to represent one or more computing devices having at least one processor (e.g., ASIC, FPGA, DSP, x86, ARM, ColdFire, GPU, multi-core processors, etc.) programmed to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable media storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, the Internet, LAN, WAN, VPN, or other type of packet switched network.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

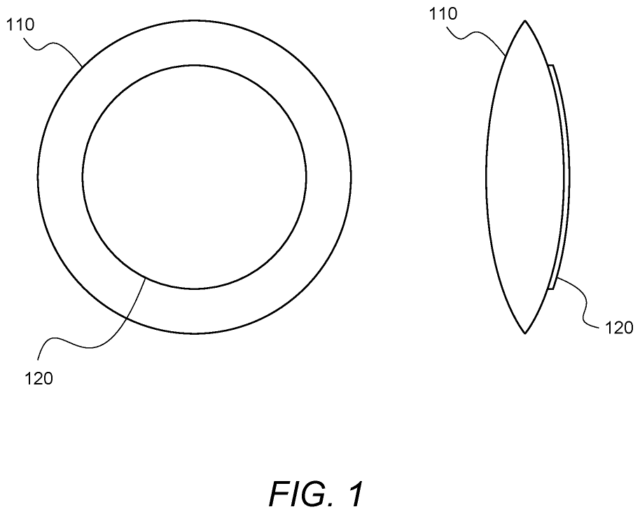

FIG. 1 shows a diagrammatic overview of a system 100 deployed within a user's eye 101, according to embodiments of the inventive subject matter.

The system 100 includes a shape-adjustable lens 110 and a transparent display 120 disposed on the shape-adjustable lens 110. The system 100 includes a processor that is communicatively connected with the transparent display 120 such that it can detect changes in the transparent display 120 and can cause the transparent display 120 to change how it displays imagery. The processor can be integral to the system 100, or externally worn by the user (such as in a pair of glasses, an earpiece, or other object worn on the body. In embodiments where the processor is external to the system 100, the device housing the processor also has communication capabilities that allows it to exchange data with the system 100 (such as a short-range wireless communication interface). As discussed herein, references to the system 100 being programmed to perform functions or carry out processes refer to the one or more processors of the system 100 and/or in communication with the system 100 carrying out the described functions and processes.

In embodiments, the system 100 includes a camera or other image sensor that is positioned to capture images of the transparent display and any images displayed thereon. In these embodiments, the system 100 is programmed to capture images of the transparent display to determine whether the image is displayed properly, as will be described further below.

In embodiments of the inventive subject matter, the system can include a camera that is disposed to capture images of the outside environment as visible from the user's eye. The system 100 is programmed to use these images for the purposes of rendering augmented reality (“AR”) overlays presented via the transparent display 120. This camera can be the same camera or a separate camera from the camera discussed above.

In embodiments of the inventive subject matter, the transparent display 120 is a display screen such as a transparent LED screen, that is capable of display images visible to the user.

In other embodiments of the inventive subject matter, the transparent display 120 is part of a display system that includes a projector. In these embodiments, the projector projects an image onto the transparent display 120, which is then visible by the user.

The system 100 can also include a power source (e.g., a battery or wireless power transmitter), and a communications interface that allows for data exchange between the system 100 and an external computing device.

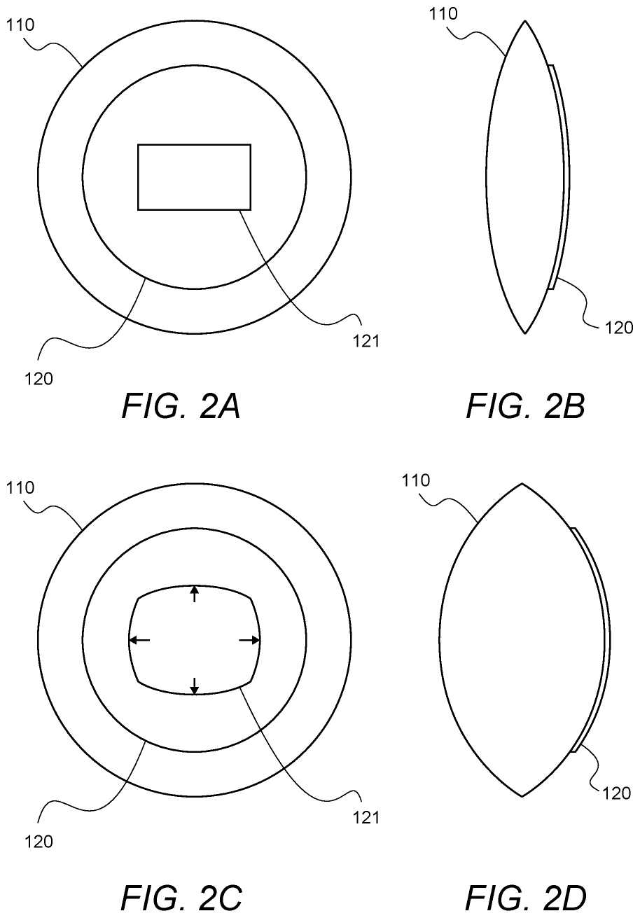

FIG. 2A shows a perspective from the view of the user of the shape-adjustable lens 110 at its default shape with the transparent display 120 showing an image 121. As seen in FIG. 2A, the image 121 has its normal rectangular shape (undistorted). When the image 121 is undistorted, the user sees it as is intended. This means that the user could read displayed text and/or view details of an image normally as displayed by the transparent display 120. This can include augmented reality elements that are overlaid over the regular “real-world” view that the user has of the outside world. FIG. 2B shows a side view of the lens 110 of FIG. 2A, with its default or “regular” shape. In the state of FIGS. 2A and 2B, the image is presented according to a default or unmodified setting.

At some point later, the shape-adjustable lens 110 changes its shape to help the user see better at a different distance. For example, the shape-adjustable lens 110 could become thicker to assist the user with their vision. As the rear surface of the shape-adjustable lens 110 changes, so does the shape of the display 120 that is attached to this rear surface. The change in the shape of the display 120 then causes an image displayed by the display 120 to become distorted. FIG. 2C shows that the change in the shape of the lens 110 and the corresponding change in the shape of the surface of the display 120 has caused the image 121 to become distorted in the direction of the arrows. FIG. 2D shows the side view of the thickened lens 110 with the display 120.

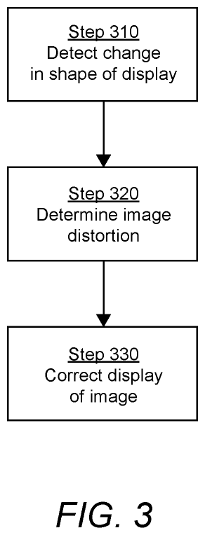

FIG. 3 is a flowchart that illustrates the processes to correct a distorted or otherwise changed image, executed according to embodiments of the inventive subject matter.

At step 310, the system 100 detects that the shape of the display 120 has changed as a result of a change in the shape of lens 110.

At step 320, the system 100 determines whether the image 121 has been distorted as a result of the change in the shape of the display 120. The distortion of the image 121 can be determined in several ways.

In embodiments where the system 100 includes a camera, the camera can capture images of the image 121 displayed by the display 120. The processor can determine, based on the captured images, whether the image 121 is distorted. This determination can be based on a comparison of a captured image against a reference image. In other embodiments, the borders or other known features of the image 121 can be analyzed to determine whether they are different from what they appear normally. For example, the processor can determine whether one or more of the borders is curved, and by how much (such as in the distortion seen in FIG. 2B). In another example, the processor can determine that the image as a whole has changed in size relative to the “normal” sized image. In yet another example, the image 121 can have reference pixels or locations that the processor knows and can locate. These reference pixels can be a known color that are then tracked by the processor as they move due to the distortion of the display 120.

In embodiments, the display screen 120 includes a sensor at one or more locations that can track the movement relative to the “at rest” state. This way, the processor can determine the degree and manner of the distortion of the display screen 120.

At step 330, the system 100 corrects the display of the distorted image 121 such that it reduces or eliminates the distortion.

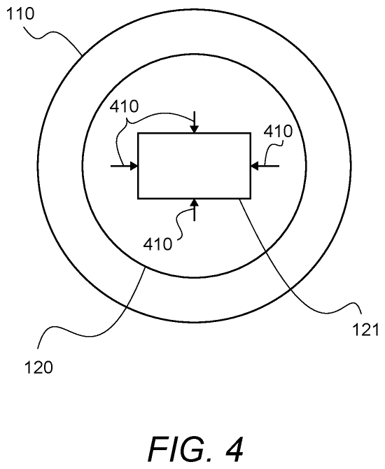

The correction by the system 100 can include an adjustment of the image 121 to account for the distortion—in essence distorting the image to counter the distortion caused by the change of the lens. For example, for the distortion seen in FIG. 2B, the processor of system 100 can adjust the horizontal dimensions to account for the curve, thus making the image appear correct to the user. This correction is shown in FIG. 4, illustrated by the arrows 410 that show the direction of movement.

Other adjustments can include changing the resolution of at least part of the image. For example, if the distortion stretches the image such that the clarity of the image decreases, the processor can increase the resolution of certain features of the image such that they appear more clear.

In embodiments, the adjustments to the image can include adjusting the brightness and/or the contrast of the image 121 to account for changes in the visibility of the image due to changes in the lens 110. In similar embodiments, the adjustments to the image can include adjusting the opacity of the displayed image or parts of the image. For example, if a distortion of the image is such that a part of the image becomes more transparent than other parts of the image, the system can adjust that distorted part of the image by making it more opaque, thus resolving the relative change in transparency.

In embodiments, the adjustments to the image can include removing part or all of the image until the lens 110 returns to its default shape.

In embodiments of the inventive subject matter, the adjustments to the image can comprise a reset or re-rendering of the image based on the new positioning/distortion of the surface area of the transparent display 120. In these embodiments, the system 100 can capture imagery from outside the eye and reset the image on the display 120 such that the image once again matches the visual environment outside of the eye. For example, if the image is an augmented reality image that is pointing to a real-world element in the environment (such as an arrow for directions that appears to be overlaid on the real-world street) and the augmented reality image is moved or otherwise distorted based on the change in shape of the lens 110, the system 100 captures the image of the real-world scene and resets the displayed image so that the augmented reality image is once again correctly positioned within the user's visual field.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A system for displaying intraocular images, comprising
a shape-adjustable lens;
a transparent display disposed on the shape-adjustable lens, the transparent display configured to display imagery; and
a processor programmed to:
cause the transparent display to display an image;
determine that the shape of the shape-adjustable lens has changed; and
adjust the display of the image on the transparent display based on the change of the shape of the shape-adjustable lens.

2. The system of claim 1, where in the processor is further programmed to adjust the display of the image based on a distortion of the image caused by a change in the shape of the shape-adjustable lens.

3. The system of claim 2, wherein the adjustment of the display of the image comprises a change of at least one of a vertical dimension and a horizontal dimension of the image.

4. The system of claim 2, wherein the adjustment of the display of the image comprises adjusting the shape of the image as displayed.

5. The system of claim 2, wherein the adjustment of the display of the image comprises adjusting the resolution of the image as displayed.

6. The system of claim 1, wherein the transparent display is disposed on a front side of the shape-adjustable lens.

7. The system of claim 1, wherein the transparent display is disposed on a rear side of the shape-adjustable lens.

8. The system of claim 1, wherein the transparent display is internal to the shape adjustable lens.

9. The system of claim 1, further comprising a camera and wherein the processor is programmed to arrange the display of the image by the transparent display based on image data received from the camera.
